(19) United States
(12) Patent Application Publication
(10) Pub. No.: US 2005/0222450 A1
(43) Pub. Date: Oct. 6, 2005

(54) Process for preparing carbamates

(75) Inventors: Sunil P. Gupte, Pune (IN); Raghunath V. Chaudhari, Pune (IN); Anandkumar Balasaheb Shivarkar, Pune (IN); Shafeek A. Mulla, Pune (IN)

Correspondence Address: DARBY & DARBY P.C., P. O. BOX 5257, NEW YORK, NY, 10150-5257, US

(73) Assignee: Council of Scientific and Industrial Research, New Delhi (IN)

(21) Appl. No.: 10/816,586

(22) Filed: Mar. 31, 2004

(51) Int. Cl. C07C 269/04

(52) U.S. Cl. 560/157

(57) ABSTRACT

A process for the preparation of carbamates from organic urea and organic carbonate in presence of a solid base catalyst is disclosed. The process works at the milder reaction conditions and utilizes inexpensive catalysts that can be recycled several times.

### FIELD OF THE INVENTION

The present invention relates to a process for preparing carbamates. Carbamates are of commercial interest as chemical intermediates in polymer industry, as herbicides in pesticide industry and as important starting material in pharmaceutical industry.

### BACKGROUND OF THE INVENTION

As is documented in the prior art, carbamates are useful as chemical intermediates in the polymer industry, as herbicides in pesticide industry and as important starting material in pharmaceutical industry.

Because of their utility, many attempts have been made to develop environmentally benign methods for preparing N-substituted carbamates. Some of the known methods involve carbonylation of amine or nitro compounds, oxidative carbonylation of amines, carboxylation reaction between amines and carbon dioxide, alcoholysis of urea and carboxylation of amines involving carbonate as carbonylating reagent. The latter two routes viz. alcoholysis of urea and carboxylation of amines by carbonate give carbamates and produce stoichiometric quantity of by-products such as amine and alcohol respectively as depicted in following reaction schemes A:

Reaction Schemes A
R₁NHCONHR₁+R₂OH→R₁NHCOOR₂+R₁NH₂ Eq I
R₁NH₂+R₂OCOOR₂→R₁NHCOOR₂+R₂OH Eq. II

The above processes suffer from several drawbacks. Carbamates are conventionally synthesized on industrial scale by phosgene route. Reference is made to Houben-Weyl, Methods of Organic Chemistry, Vol. 8, pages 137, 120 and 101, (Georg Thieme Publishers, Stuttgart, 1952), for the preparation of N-aryl carbamates on an industrial scale. Carbamates are normally produced by the reaction of alcohols with isocyanates or by the reaction of amines with chlorocarbonates. The isocyanates and chlorocarbonates used in these reactions are obtained by phosgenation of the corresponding amines or the corresponding alcohols. The drawbacks of these processes are that they are very expensive. Also, phosgene has to be used with care because of its potential danger to man and the environment.

U.S. Pat. No. 3,919,278 discloses the use of N-substituted carbamates for the manufacture of isocyanates. Reference is also made to U.S. Pat. No. 2,806,051 which describes a process whereby N-substituted carbamates are produced by reacting aniline with urea and alcohol at a mole ratio of 1.0:1.2:2.0 at temperatures below 200° C., preferably of 120° C. to 160° C. Even in the preferably used temperature range, this process results only in small yields of N-substituted carbamates if the reaction time is limited to a period which is practical in an industrial setting.

Reference is made to process described in U.S. Pat. No. 2,834,799 for making carbamic and carbonic esters by the reaction of urea with alcohols in the presence of boron trifluoride. The problem with this method is that the boron trifluoride is required in equimolar quantities so that at least one molecule of boron trifluoride is used per molecule of produced carbamic ester and at least two molecules of boron trifluoride are consumed per molecule of carbonic ester. This process is not only expensive, but it causes problems in the environment because boron trifluoride is produced in the form of the H₃N<<BF3 adduct.

Reference may be made to U.S. Pat. No. 3,449,406 for making carbamate by reacting urea with an alcohol which is in contact with an aliphatic tertiarypolyamine such as triethylene diamine etc. The carbamate yields obtained are in the range of 60-80% and the process is specific to ethylene glycol monoalkyl ether type derivatives. Further the process produces large quantity of amine as by-product, which needs to be removed to obtain carbamate in pure form.

German Published Application 21 60 111 describes a process for the manufacture of N-substituted carbamates by reacting an organic carbonate with a primary or secondary amine in the presence of a Lewis acid. The drawbacks of this process are that conversion rates are low and the reaction times are long. Furthermore, N-alkylarylamines and alcohols are always produced as by-products.

Reference may be made to U.S. Pat. No. 4,268,683 for making carbamate by reacting an amine with carbonate in presence of a catalytic quantity of Lewis acid catalyst consisting of zinc or tin compounds. The drawback of this process is that alcohols are always produced as by products and need to be removed to obtain carbamate in pure form. Reference may also be made to R. A. Franz et al. Journal of Organic Chemistry, Vol. 28, page 585 (1963), which describes a process for making methyl-N-phenyl carbamate from carbon monoxide, sulphur, aniline, and methanol. Very low yields are produced by this method; the yield does not exceed 25 percent even when there is a long reaction period.

U.S. Pat. No. 2,409,712 describes a process for making N-alkyl and N-aryl urethanes by the reaction of monoamines with urea (either N,N′- dialkyl- or N,N′-diarylurea is used) and alcohols at temperatures of 150° C. to 350° C. under increased pressure. It should be noted that this patent only describes the manufacture of N-alkylmonourethanes. U.S. Pat. No. 2,677,698 also describes a process for the manufacture of N-substituted monocarbamates. In this process, the urea is initially converted into the corresponding N,N′-disubstituted urea with monoamines, is then cleaned, and subsequently is reacted with an alcohol. The drawbacks of the process described are that it is expensive and the yields are very low. Attempts to improve the yield by improving the methods of preparing and purifying the N,N′-disubstituted ureas have been unsuccessful. Other processes have not been successful in eliminating the problems described herein. Because of the problems identified thus far, other methods of producing N-arylcarbamates have been tried.

U.S. Pat. No. 3,467,694, suggests that N-arylcarbamates can be prepared by reacting nitroaromatics with carbon monoxide, and alcohols in the presence of catalysts. Thus, carbamates may be prepared by the reaction of organic nitro compounds, carbon monoxide, and hydroxyl-containing compounds in the presence of a catalysts consisting of a noble metal and a Lewis acid under essentially anhydrous conditions in the absence of hydrogen under increased pressure and at temperatures above 150° C.

German Published Application 26 23 694 (U.S. Pat. No. 4,080,365) describes the preparation of aromatic carbamates from the hydroxyl group-containing compounds, carbon monoxide, and nitro-, nitroso-, azo- and azoxy group-containing compounds in the presence of selenium-containing catalyst systems as well as special aromatic amino and urea compounds. However, the use of these processes involve several drawbacks such as toxic carbon monoxide and catalysts, which are toxic or form toxic compounds during the reaction, such as hydrogen selenide and hydrogen sulfide, or catalysts, which are very expensive and are difficult to recycle such as palladium, require great technical expenditure and costly safety measures.

Commercial production of carbamate is almost exclusively based on phosgene technology. However, due to worldwide awareness of pollution hazards of phosgene and pollution prevention laws adopted by the Governmental agencies it is most essential to substitute it by environmentally benign routes, Therefore, it is desirable to develop a rapid and inexpensive method of preparing particular N-substituted carbamates (synonym urethane) in good yield.

### OBJECTS OF THE INVENTION

An important object of the present invention is to provide method for preparing carbamates, which obviates the drawbacks as detailed above.

Another object of the present invention is to provide a route for carbamate synthesis starting from N,N″ substituted ureas and organic carbonates.

Still another object of the present invention is to provide a non-phosgene and hence environmentally benign route for carbamate synthesis.

Yet another object of the present invention is employ a process, which utilizes environmentally benign reactants such as organic ureas and organic carbonates.

Yet another object of the present invention is to provide a process that does not produce any side products commonly encountered in reactions with amine and carbonate or urea and alcohol such as N alkylated carbamate, amines, alcohols etc.

Yet another object of the present invention is to provide a single pot reaction for the synthesis of carbamates.

Still another object of the present invention to provide a process, which uses a solid recyclable catalyst and which is stable and recyclable and not corrosive or hazardous.

Yet another object of the present invention is to provide a simple and inexpensive process for the synthesis of carbamates.

Yet another object of the present invention is to provide a simple and inexpensive process for the synthesis of carbamates which is free from solvent.

### SUMMARY OF THE INVENTION

The purpose of this invention was to produce an alkyl or aryl carbamate from readily available raw materials in one reaction stage under economically justifiable conditions with good yields. The use of strongly toxic raw materials such as phosgene, carbon monoxide, or catalysts, which are expensive or toxic and form toxic compounds during the reaction are avoided. The present invention provides a novel and improved process for preparing N-substituted carbamates by reacting urea with carbonate in the presence of a solid base catalyst at temperatures greater than 120° C. The method utilizes benign reactants such as mono alkyl/aromatic urea and carbonate and consists of a simple experimental set-up. The reaction can be run at atmospheric pressure, except in cases where organic carbonates are low boiling i.e. having a boiling point less than 120° C. The N substituted carbamates have the general formula R₁NHCOOR₂, wherein R₁ is ordinarily derived from the symmetrical or unsymmetrical urea and R₂ is derived from the organic carbonate. No prior art known to the applicants discloses the preparation of carbamates from organic ureas and organic carbonates in the presence of a catalyst.

Accordingly the present invention provides an improved process for the preparation of carbamates of general formula R₁NHCOO R₂ where R₁ and R₂ may be same or different and are selected from the group consisting of alkyl, aryl, cycloalkyl, arylalkyl and alkylaryl, which comprises reacting urea of the formula R₁NHCONHR₁ or R₁NHCONHR′₁ wherein R₁ and R′₁ are defined above, with an organic carbonate of the formula R₂OCOOR₂ or R₂OCOOR′₂ wherein R₂ and R′₂ are the or different and are selected from the group consisting of alkyl, aryl, alkylaryl and arylalkyl, at a temperature in the range of 120° C. to 200° C. for 3-12 hours in the presence of a catalytic amount of solid base catalyst under constant agitation and recovering the desired product by conventional separation.

In one embodiment of the invention, the solid base catalyst is selected from the group consisting of metal oxides, metal salt, mixed oxide, carbon, mounted base, alkali ions exchanged Zeolites and clay mineral.

In another embodiment of the invention, the clay mineral comprises Mg—Al hydrotalcite having Mg/Al ratio 2:1 to 5:1.

In an another embodiment of the invention, the metal oxide is selected from the group consisting of SiO₂—H₂O (silica gel), Al₂O₃, PbO, MgO, ZnO, ZrO₂, Na₂O and K₂O

In yet another embodiment of the invention, the metal salt is selected from the group consisting of Na₂CO₃, K₂CO₃, KHCO₃, and (NH₄)₂CO₃.

In yet another embodiment of the invention, the mixed oxide is selected from the group consisting of PbO—ZrO, PbZrO₃,SiO₂—MgO, SiO₂—CaO, SiO₂—ZnO and PbO₂—ZrO.

In yet another embodiment of the invention, the mounted base is selected from the group consisting of NaOH, KOH, K₂CO₃, alkali metal and alkaline earth metal on silica gel, alumina, and MgO.

In yet another embodiment of the invention, the alkali ion exchanged Zeolite is selected from the group consisting of Na-ZSM5, K-ZSM5, alkali impregnated zeolites, NaOH impregnated H-ZSM5, KOH impregnated H-ZSM5, and any mixture thereof.

In yet another embodiment of the invention the quantity of solid base catalyst used is in the range of 0.01-10%

In yet another embodiment of the invention the quantity of organic urea used is in the range of 0.01-80%, preferably 10-70%

In yet another embodiment of the invention the quantity of organic carbonate used is in the range of 10-90% preferably 30-90%

In yet another embodiment of the invention the organic urea is selected from the group consisting of N,N′ dimethyl urea, N,N′-p-tolylene urea, N,N′-o-Cl diphenylene urea, N,N′-m-Cl diphenylene urea, N,N′-p-Cl diphenylene urea, N,N″-p-nitro diphenylene urea, N,N′dimethyl urea, N,N′ dicyclohexyl urea and any mixture thereof

In yet another embodiment of the invention the organic carbonate is selected from the group consisting of diphenyl carbonate, dimethyl carbonate, dibutyl carbonate and any mixture thereof.

In yet another embodiment of the invention the solid catalyst used is recyclable for several time for efficient production of carbamates from organic urea and carbonate.

In yet another embodiment of the invention the carbamates obtained are N-phenyl phenyl carbamate, N-4- methylphenyl phenyl carbamate, N-2-chlorophenyl phenyl carbamate, N-3-chlorophenyl phenyl carbamate, N4-chlorophenyl phenyl carbamate, N-4-nitrophenyl phenyl carbamate, N-methyl butyl carbamate, N-phenyl methyl carbamate, N-methyl methyl carbamate and N-cylohexyl methyl carbamate.

### DETAILED DESCRIPTION OF THE INVENTION

The purpose of the present invention is to produce N-alkyl or N-aryl carbamate from readily available raw materials in one reaction step under economically justifiable conditions with good yields. The use of strongly toxic raw materials such as phosgene, carbon monoxide, or catalysts, which are expensive or toxic and form toxic compounds during the reaction have been avoided. Alcoholysis of urea and carboxylation of amines by carbonate give carbamates and produce stoichiometric quantity of by-products such as amine and alcohol respectively as depicted in reaction scheme A. The problems encountered in the prior art methods have been solved by the process of the present invention which is able to produce preparation of the alkyl and aryl carbamates utilizing benign reactants such as alkyl and/or aromatic urea and organic carbonate in the presence of a catalyst at temperatures greater than 120° C. utilizing a simple set-up. The reaction can be run at atmospheric pressure, except in cases where organic carbonates are low boiling i.e., having boiling points less than 120° C. The exact mechanism of the reaction is not completely understood but catalyst is essential in order to obtain the desired N-substituted carbamates from these reactants in excellent yield. The N- substituted carbamates have the general formula R₁—NH—CO—O R₂, wherein R₁ is ordinarily derived from the disubstituted symmetrical or unsymmetrical urea and R₂ is derived from the organic carbonate. The reaction may be illustrated by the reaction scheme Al as follows:

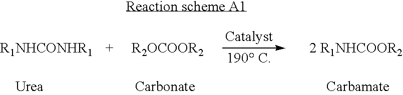

The organic urea may be any symmetrical or unsymmetrical urea. Symmetrical urea is defined by general formula R₁NHCONH R₁ wherein R₁ is selected from an alkyl group, cycloalkyl group, aryl group, arylalkyl group and alkylaryl group. Where R₁ is alkyl it can be methyl, ethyl, n-propyl, isopropyl, n-butyl, isobutyl, n-octyl, isooctyl, and the like. Where R₁ is a methyl group this include N,N′ dimethyl urea and the like. Where R₁ is a cycloalkyl group it may be cyclopropyl, cyclobutyl, cyclohexyl and the like. Where R₁ is cyclohexyl group this include N,N′ dicyclohexyl urea and the like. Where R₁ is aryl group it may be phenyl, napthyl, anthryl and the like. Where R₁ is phenyl group this include N,N″diphenyl urea and the like. Where R₁ is arylalkyl group it can be phenylmethyl, phenylethyl, phenylpropyl and the like. Where, R₁ is phenylmethyl group this include N,N″dibenzyl urea and the like. Where R₁ is alkylaryl group it can be methylphenyl, ethylphenyl, propylphenyl and the like. Where R₁ is methylphenyl group this includes N′,Sr—Bis(methylphenyl) urea and the like. Examples of the substituents for the alkyl group, aryl group, arylalkyl group and alkylaryl group include hydroxyl, alkoxyl, nitro and halogen groups and the like. Where R₁ is substituted e.g., chloro substitution to phenyl group this include R₁N′ Bistchlorophenyl urea and the like. Unsymmetrical urea is defined by general formula R₁NHCONHR′₁ where, R₁ and R′₁ are the same or different. Examples of unsymmetrical urea include wherein R₁ and R′₁ are alkyl group, aryl group, alkylaryl group and arylalkyl group, for example R₁ and R′₁ are alkyl the they can be methyl, ethyl, propyl, isopropyl, butyl, isobutyl, octyl, isooctyl, and the like. Examples of unsymmetrical ureas are where R₁ is methyl group and R′₁ is ethyl group. This includes N methyl N′ ethyl urea and the like. Where R₁ and R′₁ are substituted alkyl group, aryl group, alkylaryl group and arylalkyl group, the substituents include halogen, hydroxy, alkoxy, nitro, alkyl, aryl, napthyl. Example of substitution on phenyl group is for example halogen, where R₁ is chlorophenyl and R′₁ is bromophenyl. This includes N-(chlorophenyl) N′-(bromophenyl) urea and the like. Intra group combination can also be employed e.g. R₁ is from alkyl e.g. methyl and R′₁ from aryl e.g. phenyl. This includes N-methyl N″-phenyl urea and like. Mixtures of ureas can also be employed.

The organic carbonate can be a symmetrical or unsymmetrical carbonate. Symmetrical organic carbonate can be any carbonate of the general formula R₂OCOOR₂ wherein R₂ is alkyl group, aryl group, alkylaryl group and arylalkyl group. Where R₂ is alkyl group it can be methyl, ethyl, propyl, isopropyl, butyl, isobutyl, octyl, isooctyl, and the like. Where R₂ is methyl this includes most common form of organic carbonate, viz. dimethyl carbonate. Where R₂ is aryl group it can be phenyl, napthyl, anthryl and the like. Where R₂ is phenyl group this includes diphenyl cabonate. Where R₂ is arylalkyl group it may be phenylmethyl, phenylethyl, phenylpropyl and the like. Where R₂ is phenylmethyl group this includes dibenzyl carbonate and the like. Where R₂ is alkylaryl group it may be methylphenyl, ethylphenyl, propylphenyl and the like. Where R₂ is methylphenyl group this includes Bis(methyl phenyl) carbonate and the like, Examples of the substituents on the alkyl group, aryl group, arylalkyl group and alkylaryl group includes hydroxyl, alkoxyl, nitro and halogen groups and the like. Where R₂ is substituted e.g. chloro substitution to phenyl group this includes Bis(chlorophenyl) carbonate and the like. Unsymmetrical carbonate is defined by general formula R₂OCOOR′₂ where, R₂ and R′₂ are the same or different. Examples of unsymmetrical carbonate include compounds wherein R₂ and R′₂ is alkyl group, aryl group, alkylaryl group and arylalkyl group. For example if R₂ and R′₂ are alkyl they can be methyl, ethyl, propyl, isopropyl, butyl, isobutyl, octyl, isooctyl, and the like. Examples of unsymmetrical carbonate are where R₂ is methyl group and R′₂ is ethyl group. This includes methyl ethyl carbonate and like. Where R₂, R′₂ are substituted alkyl group, aryl group, alkylaryl group and arylalkyl group the substituents can be halogen, hydroxyl, alkoxyl, nitro, alkyl, aryl, napthyl. An example of substitution on phenyl group is halogen. Where R₂ is chlorophenyl and R′₂ is bromophenyl, this includes chlorophenyl bromophenyl carbonate and the like. Intra group combination can also be employed e.g., R₂ is from alkyl e.g. methyl and R′₂ from aryl e.g. phenyl. This includes methyl phenyl carbonate and like. Mixtures of carbonates can also be employed.

The reaction between unsymmetrical urea and unsymmetrical carbonate yield four possible unsymmetrical carbamates which may be illustrated by reaction scheme A2 as follows:

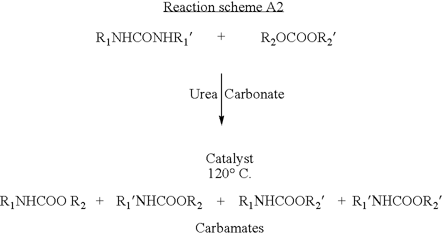

In the present invention the reactants can be employed on an equimolar basis or one may be present in excess of the other up to about 50, preferably one to about 5 mols in excess of the other. It is preferred that the organic carbonate reactant be employed in excess compared to urea. Reaction may be carried out employing an inert solvent such as high boiling alkanes for example decane, paraffins etc., substituted inert aromatics such as mono chloro benzenes, dichloro benzenes etc.

The reaction should be conducted under the condition of vigorous stirring so that all the reactants and catalyst remain in well-mixed state and catalyst is well suspended in liquid phase. Generally a stirrer speed in the range of 100-1500 revolution per minute (rpm) is employed and more preferably, stirrer speed in the range 500-1,000 rpm is necessary.

The present invention is generally carried out at atmospheric pressure or the autogenous pressure of the reaction system, although, higher pressure up to 50 atmospheres by means of filling the reactor by gases such as nitrogen, argon or carbon dioxide may be employed. This is especially so at higher reaction temperatures or when the reaction temperature is above boiling temperature of the reactants.

In the present invention the solid base catalyst can be recycled several times in the process so as to increase the efficiency of the catalyst and productivity of carbamate formation.

The present invention will now be described in greater detail wit reference to the following Examples, which are given merely by way of illustration and therefore, should not be construed to limit the scope of the present invention.

### EXAMPLE-1

3.16×10″³ mol of N,N′ diphenyl urea, 15.56×10³ mol diphenyl dicarbonate and 200 mg HaO (W. R. Grace make silica gel) catalyst were charged to a well flushed and dried 3-necked round bottom reaction vessel (50 cc) equipped with a thermometer, stirrer and reflux condenser. The contents were heated under stirring up to 150° C. and kept for 8 hours while inert atmosphere was maintained. After cooling to room temperature the solid mass was dissolved in acetone, filtered to separate the catalyst and reaction crude. N-phenyl phenyl carbamate was isolated in pure form by column chromatography (silica gel, ethyl acetate-chloroform 0.2:9.8) and characterized by elemental analysis, ¹H NMR, ¹³C NMR, IR. Products and unconverted reactants were analyzed by liquid chromatography (LC) for conversion of N,N′ diphenyl urea and organic dicarbonate and selectivity to carbamate. Urea being the limiting reactant in this case conversion of urea was calculated on the basis of moles of urea consumed by the reaction. N-substituted carbamate selectivity was calculated based on the urea consumed according to stoichiometry shown in reaction scheme Al. Analysis of reaction crude by LC showed conversion of N,N′ diphenyl urea to be 100% and selectivity to N-phenyl carbamate to be 96%.

### EXAMPLE-2

The procedure in example-1 was exactly repeated except that the catalyst, which was recovered as shown in Exmple-1, was washed with acetone and dried at 100° C. for six hours and was charged to the reactor along with 3.16×10˜³ mol N,N′diphenyl urea, 15.56×10″³ mol diphenyl carbonate. After cooling to room temperature the LC analysis of reaction crude showed 78% diphenyl urea conversion and selectivity to N-phenyl phenyl carbamate to be 98%.

### EXAMPLE-3

The procedure in example-1 was exactly repeated except that for the charge 3.16×10″³ mol N,N′diphenyl urea, 15.56×10′³ mol diphenyl carbonate and recovered catalyst of Example-2, was washed with acetone and dried at 100° C. for six hours and was charged to the reactor.

After cooling to room temperature the LC analysis of reaction crude showed 79% diphenyl urea conversion and selectivity to N-phenyl phenyl carbamate to be 98%.

### EXAMPLE-4

The procedure in example-1 was exactly repeated except that for the charge 3.16×10˜³ mol N,N′diphenyl urea, 15.56×10″³ mol diphenyl carbonate and recovered catalyst of Example-3, was washed with acetone and dried at 100° C. for six hours and was charged to the reactor. After cooling to room temperature the LC analysis of reaction crude showed 77% diphenyl urea conversion and selectivity to N-phenyl phenyl carbamate to be 98%.

### EXAMPLE-5

The procedure in example-1 was exactly repeated except that for the charge 3.16×10″³ mol N,N′diphenyl urea, 15.56×10″³ mol diphenyl carbonate and recovered catalyst of Example-4, was washed with acetone and dried at 100° C. for six hours and was charged to the reactor. After cooling to room temperature the LC analysis of reaction crude showed 72% diphenyl urea conversion and selectivity to N-phenyl phenyl carbamate to be 99%.

### EXAMPLE-6

The procedure in example-1 was exactly repeated except that for the charge 3.16×10″³ mol N,N′diphenyl urea, 15.56×10″³ mol diphenyl carbonate and recovered catalyst of Example-5, was washed with acetone and dried at 100° C. for six hours and was charged to the reactor. After cooling to room temperature the LC analysis of reaction crude showed 68% diphenyl urea conversion and selectivity to N-phenyl phenyl carbamate to be 98%.

### EXAMPLE-7

The procedure in example-1 was exactly repeated except that for the charge 3.16×10˜³ mol N,NMiphenyl urea, 15.56×10″³ mol diphenyl carbonate and recovered catalyst of Example-6, was washed with acetone and dried at 100° C. for six hours and was charged to the reactor. After cooling to room temperature the LC analysis of reaction crude showed 61% diphenyl urea conversion and selectivity to N-phenyl phenyl carbamate to be 98%.

### EXAMPLE-8

The procedure in example-1 was exactly repeated except that the catalyst, which was recovered as shown in Exmple-1, was washed with acetone, dried and calcined at 500° C. for six hours, cooled and was charged to the reactor along with 3.16×10″³ mol N,N′diphenyl urea, 15.56×10″³ mol diphenyl carbonate. After cooling to room temperature the LC analysis of reaction crude showed 97% diphenyl urea conversion and selectivity to N-phenyl phenyl carbamate to be 98%.

### EXAMPLE-9

The procedure in example-1 was exactly repeated except that for the charge 3.16×10″ mol N,N′diphenyl urea, 15.56×10˜³ mol diphenyl carbonate and recovered catalyst from Example-8, was washed with acetone, dried and calcined at 500° C. for six hours and was charged to the reactor. After cooling to room temperature the LC analysis of reaction crude showed 93% diphenyl urea conversion and selectivity to N-phenyl phenyl carbamate to be 99%.

### EXAMPLE-10

The procedure in example-1 was exactly repeated except that for the charge 3.16×10″³ mol N,N′diphenyl urea, 15.56×10˜³ mol diphenyl carbonate and recovered catalyst of Example-9, was washed with acetone, dried and calcined at 500° C. for six hours and was charged to the reactor. After cooling to room temperature the LC analysis of reaction crude showed 96% diphenyl urea conversion and selectivity to N-phenyl phenyl carbamate to be 98%.

### EXAMPLE-11

The procedure in example-1 was exactly repeated except that for the charge 3.16×10˜³ mol N,N′diphenyl urea, 15.56×10″ mol diphenyl carbonate and recovered catalyst of Example-10, was washed with acetone, dried and calcined at 500° C. for six hour and was charged to the reactor. After cooling to room temperature LC analysis of reaction crude showed 93% diphenyl urea conversion and selectivity to N-phenyl phenyl carbamate of 98%.

### EXAMPLE-12

The procedure in example-1 was exactly repeated except that for the charge 3.16×10″³ mol N,N′diphenyl urea, 15.56×10″³ mol diphenyl carbonate and recovered catalyst of Example-11, was washed with acetone, dried and calcined at 500° C. for six hours and was charged to the reactor. After cooling to room temperature LC analysis of reaction crude showed 89% diphenyl urea conversion and selectivity to N-phenyl phenyl carbamate of 98%.

### EXAMPLE-13

The procedure in example-1 was exactly repeated except that for the charge 3.16×10″³ mol N,N′diphenyl urea, 15.56×10″ mol diphenyl carbonate and 200 mg Davisil silica gel catalyst were added. After cooling to room temperature the LC analysis of reaction crude showed 25% N,N^{X} diphenyl urea conversion and selectivity to N-phenyl phenyl carbamate to be 97%.

### EXAMPLE-14

The procedure in example-1 was exactly repeated except that for the charge 3.16×10″ mol diphenyl urea, 15.56×10″³ mol N,N′diphenyl carbonate and 200 mg 5% Pb on silica gel catalyst were added. After cooling to room temperature the LC analysis of reaction crude showed 21% diphenyl urea conversion and selectivity to N-phenyl phenyl carbamate to be 98%.

### EXAMPLE-15

The procedure in example-1 was exactly repeated except that for the charge 3.16×10˜³ mol diphenyl urea, 15.56×10˜³ mol N,N′diphenyl carbonate and 0.9×10″³ mol PbZrO₃ catalyst were added. After cooling to room temperature LC analysis of reaction crude showed 99% diphenyl urea conversion and selectivity to N-phenyl phenyl carbamate of 93%.

### EXAMPLE-16

The procedure in example-1 was exactly repeated except that for the charge 3.16×10″³ mol diphenyl urea, 15.56×10˜³ mol N,N′diphenyl carbonate and 200 mg Mg—AL hydrotalcite (Mg/Al ratio 3:1) catalyst were added. After cooling to room temperature the LC analysis of reaction crude showed 99% diphenyl urea conversion and selectivity to N-phenyl phenyl carbamate to be 95%.

### EXAMPLE-17

The procedure in example-1 was exactly repeated except that for the charge 3.16×10˜³ mol diphenyl urea, 15.56×10″³ mol N,N′diphenyl carbonate and 200 mg Na-ZSM-5 (Si/Al=130) catalyst were added. After cooling to room temperature the LC analysis of reaction crude showed 80% diphenyl urea conversion and selectivity to N-phenyl phenyl carbonate to be 98%.

### EXAMPLE-18

The procedure in example-1 was exactly repeated except that the catalyst, which was recovered at the end of reaction from Exmple-17, was washed with acetone, dried and calcined at 500° C. for six hours, cooled and was charged to the reactor along with 3.16×10″³ mol N\hat{ }diphenyl urea, 15.56×10″³ mol diphenyl carbonate. After cooling to room temperature the LC analysis of reaction crude showed 79% diphenyl urea conversion and selectivity to N-phenyl phenyl carbamate to be 98%.

### EXAMPLE-19

The procedure in example-1 was exactly repeated except that for the charge that 3.16×10″³ mol N,N′diphenyl urea, 15.56×10˜³ mol diphenyl carbonate and recovered catalyst of Example-18, was washed with acetone, dried and calcined at 500° C. for six hours and was charged to the reactor. After cooling to room temperature the LC analysis of reaction crude showed 78% diphenyl urea conversion and selectivity to N-phenyl phenyl carbamate to be 98%.

### EXAMPLE-20

The procedure in example-1 was exactly repeated except that for the charge that 3.16×10˜³ mol N,lSrdiphenyl urea, 15.56×10″³ mol diphenyl carbonate and recovered catalyst of Example-19, was washed with acetone, dried and calcined at 500° C. for six hours and was charged to the reactor. After cooling to room temperature the LC analysis of reaction code showed 86% diphenyl urea conversion and selectivity to N-phenyl phenyl carbamate to be 97.5%.

### EXAMPLE-21

The procedure in example-1 was exactly repeated except that for the charge that 3.16×10″³ mol N,N′diphenyl urea, 15.56×10″ mol diphenyl carbonate and recovered catalyst of Example-20, was washed with acetone, dried and calcined at 500° C. for six hours and was charged to the reactor. After cooling to room temperature LC analysis of reaction crude showed 83% diphenyl urea conversion and selectivity to N-phenyl phenyl carbamate of 98%.

### EXAMPLE-22

The procedure in example-1 was exactly repeated except that for the charge that 3.16×10*³ mol N,N″diphenyl urea, 15.56×10′³ mol diphenyl carbonate and recovered catalyst of Example-21, was washed with acetone, dried and calcined at 500° C. for six hours and was charged to the reactor. After cooling to room temperature LC analysis of reaction crude showed 77% diphenyl urea conversion and selectivity to N-phenyl phenyl carbamate of 98%.

### EXAMPLE-23

The procedure in example-1 was exactly repeated except that for the charge 3.16×10″³ mol diphenyl urea, 15.56×10″³ mol N,N′diphenyl carbonate and 200 mg Na-ZSM-5 (Si/Al=55) catalyst were added and reaction run for 15 hours. After cooling to room temperature the LC analysis of reaction crude showed 62% diphenyl urea conversion and selectivity to N-phenyl phenyl carbamate to be 98%.

### EXAMPLE-24

The procedure in example-1 was exactly repeated except that for the charge 3.16×10˜³ mol diphenyl urea, 15.56×10″³ mol N,N′diphenyl carbonate and 200 mg 5% Zn on silica gel (W. R. Grace, USA) catalyst was added. After cooling to room temperature the LC analysis of reaction crude showed 67% diphenyl urea conversion and selectivity to N-phenyl phenyl carbamate to be 98%.

### EXAMPLE-25

The procedure in example-1 was exactly repeated except that for the charge 3.16×10″ mol diphenyl urea, 15.56×10˜³ mol N,N′diphenyl carbonate and 200 mg Li—MgO, catalyst were added. After cooling to room temperature the LC analysis of reaction crude showed 92% diphenyl urea conversion and selectivity to N-phenyl to phenyl carbamate to be 98%.

### EXAMPLE-26

The procedure in example-1 was exactly repeated except that for the charge 3.16×10″ mol diphenyl urea, 15.56×10-3 mol N,N′diphenyl carbonate and 0.9×10″³ mol Mg(OH)₂ catalyst were added. After cooling to room temperature the LC analysis of reaction crude showed 68% diphenyl urea conversion and selectivity to N-phenyl phenyl carbamate to be 99%.

### EXAMPLE-27

The procedure in example-1 was exactly repeated except that for the charge 3.16×10″ mol diphenyl urea, 15.56×10″ mol N,N′diphenyl carbonate and 0.9×10 mol K\hat{ }COs catalyst were added and reaction run for 15 hours. After cooling to room temperature the LC analysis of reaction crude showed 30% diphenyl urea conversion and selectivity to N-phenyl phenyl carbamate of 98%.

### EXAMPLE-28

The procedure in example-1 was exactly repeated except that for the charge 3.16×10″³ mol diphenyl urea, 15.56×10″³ mol N,N′diphenyl carbonate and 0.9×10˜³ mol PbO catalyst were added and reaction run, for 15 hours. After cooling to room temperature LC analysis of reaction crude showed 96% diphenyl urea conversion and selectivity to N-phenyl phenyl carbamate of 99%.

### EXAMPLE-29

The procedure in example-1 was exactly repeated except that for the charge 3.16×10″³ mol -t T diphenyl urea, 15.56×10 mol N,N′diphenyl carbonate and 0.9×10 mol PbCOs catalyst were added and reaction run for 15 hours. After cooling to room temperature the LC analysis of reaction crude showed 87% diphenyl urea conversion and selectivity to N-phenyl phenyl carbamate to be 96%.

### EXAMPLE-30

The procedure in example-1 was exactly repeated except that for the charge 3.16×10″ mol N,N′diphenyl urea, 15.56×10″³ mol diphenyl carbonate and 200 mg A\hat{ }Oa neutral catalyst (activated Brockman-1, USA) were added. A cooling to room temperature the LC analysis of reaction crude showed 12% diphenyl urea conversion and selectivity to N-phenyl phenyl carbamate to be 97%.

### EXAMPLE-31

The procedure in example-1 was exactly repeated except that for the charge 3.16×10″³ mol N,N′-Bis(4-methylphenyl) urea, 15.56×10″³ mol diphenyl carbonate and 200 mg W. R. Grace silica gel catalyst were added. After cooling to room temperature LC analysis of reaction crude showed 92% diphenyl urea conversion and selectivity to N4-methylphenyl phenyl carbamate to be 99%.

### EXAMPLE-32

The procedure in example-1 v/as exactly repeated except that for the charge 3.16×10″³ mol N,N″-Bis(2-chlorophenyl) urea, 15.56×10″³ mol diphenyl carbonate and 200 mg W. R. Grace silica gel catalyst were added. After cooling to room temperature the LC analysis of reaction crude showed 76% diphenyl urea conversion and selectivity to N-2-chlorophenyl phenyl carbamate to be 97%.

### EXAMPLE-33

The procedure in example-1 was exactly repeated except that for the charge 3.16×10″ mol N,N′-Bis(3-chlorophenyl) urea, 15.56×10″³ mol diphenyl carbonate and 200 mg W. R. Grace silica gel catalyst were added. After cooling to room temperature LC analysis of reaction crude showed 85% diphenyl urea conversion and selectivity to N-3-chlorophenyl phenyl carbamate selectivity to be 99%.

### EXAMPLE-34

The procedure in example-1 was exactly repeated except that for the charge 3.16×10″³ mol N,N′-Bis(4-chlorophenyl) urea, 15.56×10″³ mol diphenyl carbonate and 200 mg W. R. Grace silica gel catalyst were added. After cooling to room temperature the LC analysis of reaction crude showed 94% diphenyl urea conversion and selectivity to N-4-chlorophenyl phenyl carbamate to be 99%.

### EXAMPLE-35

The procedure in example-1 was exactly repeated except that for the charge 3.16×10″³ mol N,N′-Bis(4-nitrophenyl) urea, 15.56×10″ mol diphenyl carbonate and 200 mg W. R. Grace silica gel catalyst were added. After cooling to room temperature the LC analysis of reaction crude showed 94% diphenyl urea conversion and selectivity to N-4-nitrophenyl phenyl carbamate to be 98%.

### EXAMPLE-36

The procedure in example-1 was exactly repeated except that for the charge 3.16×10″ mol N,N′dimethyl urea, 15.56×10″³ mol dibutyl carbonate and 200 mg 5% K-silica gel catalyst were added. After cooling to room temperature the LC analysis of reaction crude showed 87% diphenyl urea conversion and selectivity to N-methyl butyl carbamate to be 98%.

### EXAMPLE-37

The procedure in example-1 was exactly repeated except that no catalyst was added. After cooling to room temperature the LC analysis of reaction crude showed 1% diphenyl urea conversion and selectivity to N-phenyl phenyl carbamate to be 88%.

### EXAMPLE-38

A 50 ml capacity stainless steel high pressure reactor equipped with gas and liquid charging and sampling valves, temperature control, cooling water and a turbined blade stirrer was charge with 6.32×10″³ mol diphenyl urea, 15 ml dimethyl carbonate and 400 mg W. R. Grace silica gel catalyst. The reactor was flushed twice with nitrogen and pressurized with nitrogen up to 500 psig pressure. The contents were heated at 150° C. and vigorously stirred for 12 hours. After cooling to room temperature the reaction mixture was filtered to separate the catalyst. Analysis of reaction crude by LC showed conversion of N,N″ diphenyl urea to be 22% and selectivity to N-phenyl methyl carbamate to be 97%.

### EXAMPLE-9

The procedure in example-38 was exactly repeated except that for the charge 6.32×10″³ mol N,N′dimethyl urea, 15 ml dimethyl carbonate and 400 mg W. R. Grace silica gel catalyst were added. After cooling to room temperature the reaction mixture was filtered to separate the catalyst. Analysis of reaction crude by LC showed conversion of N,N′ diphenyl urea to be 91% and selectivity to N-methyl methyl carbamate to be 98%.

### EXAMPLE-40

The procedure in example-38 was exactly repeated except that for the charge 6.32×10″³ mol N,N′ dicyclohexyl urea, 15 ml dimethyl carbonate and 400 mg W. R. Grace silica gel catalyst were added. After cooling to room temperature the reaction mixture was filtered to separate the catalyst. Analysis of reaction crude by LC showed conversion of N,N′ diphenyl urea to be 91% and selectivity to N-cylohexyl methyl carbamate to be 97%.

### EXAMPLE-41

The procedure in example-38 was exactly repeated except that for the charge 6.33×10″³ N,N′ diphenyl urea, 5.58×10′² mol diphenyl carbonate and 400 mg W. R. Grace silica gel catalyst were added. After cooling to room temperature the reaction mixture was filtered to separate the catalyst. Analysis of reaction crude by LC showed conversion of N,N′ diphenyl urea to be 75% and selectivity to N-phenyl phenyl carbamate to be 97%.

Advantageous Features of the Present Invention

1. Present invention for the first time demonstrates the preparation of carbamates starting from environmentally benign reactants such as organic urea and carbonate.
2. The method utilizes a solid base catalyst, which is highly efficient and recyclable.
3. The process consists of a simple experimental set-up and uses inexpensive catalyst
4. The method does not produce any side product and hence 100% atom utility can be achieved.

1. A process for the preparation of carbamates of general formula R₁NHCOOR₂ where R₁, and R₂ may be same or different which comprises reacting urea having the formula R₁NHCONHR₁, or R₁NHCONHR′₁ wherein R₁ and R′₁, are selected from alkyl, aryl, cycloalkyl, arylalkyl and alkylaryl, with an organic carbonate having the formula R₂OCOOR₂ or R₂0COOR′₂ wherein R′₂ and R′₂ may be same or different and are selected from alkyl, aryl, alkylaryl and arylalkyl, at a temperature in the range of 120° C. to 200° C. in the presence of a catalytic amount of a solid base catalyst under constant agitation and recovering the desired product by conventional separation methods.

2. A process as claimed in claim 1 wherein said reaction is carried out for a period of 3 to 12 hrs.

3. A process as claimed in claim 1, wherein said solid base catalyst is selected from the group consisting of metal oxides, metal salt, mixed oxide, carbon, mounted base, alkali ions exchanged Zeolites and clay mineral such as Mg—Al hydrotalcite having Mg/Al ratio 2:1 to 5:1.

4. A process as claimed in claim 3 wherein said metal oxide is selected from the group consisting of SiO₂<<H₂O (silica gel), Al₂C>3, PbO, MgO, ZnO₂, ZrO, Na₂O and K₂O.

5. A process as claimed in claim 3 wherein said metal salt is selected from the group consisting of Na₂CC>3, K\hat{ }CCb, KHCOa, and (NH₄)2CO₃.

6. A process as claimed in claim 3 wherein said mixed oxide is selected from the group consisting of PbO—ZrO, PbZrO₃, SiO₂—MgO, SiO₂—CaO, SiO₂—ZnO and PbO₂—ZrO.

7. A process as claimed in claim 3 wherein said mounted base is selected from the group consisting of NaOH, KOH, K₂CC>3, alkalimetal and alkaline earth metal on silica gel, alumina, and MgO.

8. A process as claimed in claim 3 wherein said alkali ions exchanged Zeolites are selected from the group consisting of Na or K-ZSM-5 and/or alkali impregnated zeolites, NaOH or KOH impregnated H-ZSM-5.

9. A process as claimed in claim 1 wherein said solid base catalyst is employed in an amount of from 0.01-10%.

10. A process as claimed in claim 9, wherein said solid base catalyst is employed in an amount of from 0.01-80%, preferably, 10-70%.

11. A process as claimed in claim 1, wherein said organic carbonate is employed in an amount in the range of from 10 to 90%, preferably 30 to 90%.

12. A process as claimed in claim 1, wherein the organic urea is selected from the group consisting of N,N¹ dimethyl urea, N,N′p-tolylene urea, N,N^{r}-o-Cl diphenylene urea, N,N′-m-Cl diphenylene urea, N,N′p-Cl diphenylene urea, N,N″p-nitro diphenylene urea, N,N′ dimethyl urea, N,N′ dicyclohexyl urea and any mixture thereof.

13. A process as claimed in claim 1, wherein said organic carbonate used is selected from the group consisting of diphenyl carbonate, dimethyl carbonate, dibutyl carbonate and mixture thereof.

14. A process as claimed in claim 1 wherein said solid catalyst is recyclable several times for efficient production of carbamates from organic urea and carbonate.

15. A process as clamed in claim 1, wherein carbamate obtained are N-phenyl phenyl carbamate, N-4-methylphenyl phenyl carbamate, N-2-chlorophenyl phenyl carbamate, N-3-chlorophenyl phenyl carbamate, N-4-chlorophenyl phenyl carbamate, N-4-nitrophenyl phenyl carbamate, N-methyl butyl carbamate, N-phenyl methyl carbamate, N-methyl methyl carbamate and N-cylohexyl methyl carbamate.
